(19) United States
(12) Patent Application Publication
(10) Pub. No.: US 2007/0077202 A1
(43) Pub. Date: Apr. 5, 2007

(54) Histostain composition for endoscope

(75) Inventors: Akira Yamamoto, Tokyo (JP); Yusuke Iimori, Tokyo (JP); Mae Koyama, Tokyo (JP); Mariko Ishiguro, Tokyo (JP); Mizue Saze, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/633,567

(22) Filed: Dec. 5, 2006

(30) Foreign Application Priority Data
Jun. 12, 2005 (JP) 2005-352143

(51) Int. Cl. A61K 49/00

(52) U.S. Cl. 424/9.1; 424/9.6

(57) ABSTRACT

The invention relates to a histostain composition for endoscope containing one or more members selected from colors derived from Monascus. The stain composition is a staining agent which sharpens the shapes of digestive tract lumen surfaces and the like with a light in the visible wavelength range, having a function of being excited by a light of specific wavelength to emit fluorescence, and being biologically safe and suitable for endoscopy.

### FIELD OF THE INVENTION

The present invention relates to a histostain composition used in diagnosis with an endoscope.

### DESCRIPTION OF THE RELATED ART

Diagnostic techniques using an endoscope are wide-spreading and are applied to gastrointestinal endoscopy in upper and lower digestive tracts and particularly to diagnosis of disorders such as cancer, peptic ulcer, peptic colitis, and the like. Detection of a histological abnormality (affected region) by endoscopic examination is conducted generally using an endoscope (magnification of about 10-power to 500-power) under a visible light without using a staining agent. On one hand, there is a method called dye spraying endoscopy wherein the surface of tissue is sprayed with a dye-containing solution and observed with an endoscope. By this dye spraying endoscopy, the form of the surface of a digestive tract lumen can be clearly observed, and thus a minute affected region can be easily observed owing to a change in color tone. The endoscope used in the dye spraying endoscopy includes a visible light endoscope and a fluorescent endoscope.

The dye used mainly in the dye spraying endoscopy is for example indigo carmine for staining a digestive tract lumen under a visible light and fluorescein for fluorescence staining.

For diagnosis, it is important to observe not only the surface of tissue in the living body but also the interior of tissue in the living body. In a general method of observing the interior of tissue in the living body, a micropart of tissue obtained by biopsy is cut thin in a laboratory, then stained and observed. In a method of observing the interior of biological tissues in situ, MRI, PET, CT, soft X-ray method and the like are applied for observation of the whole body. For gastrointestinal endoscopy, an endoscope to which the self-fluorescence reaction of biological tissue is applied has been commercialized. By irradiating the biological tissue with a light of specific wavelength, self-fluorescence is generated by an endogenous substance of the tissue, and thus a normal region and an affected region can be optically observed and distinguished owing to a difference in intensity and its spectrum.

In usual endoscopic observation, however, it is inevitable that the affected region is empirically judged and a tissue fragment is excised and separately diagnosed by techniques such as histological staining in a laboratory. According to a recently developed confocal endoscope, on the other hand, the interior of tissue can be observed without excising the tissue. That is, the confocal system is a technique in which a light reflected only by the in-focus surface of the tissue is detected through a pinhole arranged before a detector, whereby a clear image can be obtained. Commonly, in the confocal optical system, a fluorescent image of a sample stained with a fluorescent substance is observed by scanning the stained sample with a laser light. Accordingly, a fluorescent dye is necessary. A confocal endoscope adopting the confocal system has both an ordinary monitoring optical system and a confocal monitoring optical system and is thus useful in that the screening of an affected region and the optical observation by optical thin cutting of tissue without excising cells are made feasible in situ with less invasiveness.

As the fluorescent dye used for the purpose of interstitial observation with a confocal endoscope, fluorescein and acriflavine are known from a literature (Gastroenterology 2004, Vol. 127, No. 3, pp. 706-713). In this literature, a large amount of fluorescein is intravenously administered, and when fluorescein reaches the digestive tract tissue, interstitial observation with a confocal endoscope is conducted. In the case of acriflavine, this dye is sprayed directly onto the digestive tract prior to interstitial observation, but fails to give a clear stained image, and thus it is described therein that fluorescein is more useful than acriflavine.

### SUMMARY OF THE INVENTION

However, there is a problem that the fluorescein dye used conventionally in dye spraying endoscopy has serious side effects. In the case of acriflavine, its side effect on the living body is problematic because it is an antibiotic. In dye spraying endoscopy, particularly confocal endoscopy, there is demand for dyes capable of staining cells in a short time, sharpening the shape of tissue surface for observation with a light source of either visible light or fluorescence excitation light, and further staining the interior of tissue.

Accordingly, the object of the invention is to provide a staining agent which sharpens the shapes of digestive tract lumen surfaces and the like under a light in the visible wavelength range, having a function of being excited by a light of specific wavelength to emit fluorescence, and being biologically safe and suitable for endoscopy.

From the viewpoint of safety, staining property under a visible light, and staining property under fluorescence, the present inventors paid attention to natural colors, and as a result of extensive study, they unexpectedly found that colors derived from Monascus are characterized by being excellent in staining property under a visible light, having fluorescence whose wavelength being different from its excitation wavelength, being useful not only as a staining agent in usual endoscopy but also as a fluorescent dye for interstitial staining in confocal endoscopy, to give a vivid stained image useful in detection of a small affected region, and staining only the cytoplasm without staining cell nuclei, thus indicating that these colors have reduced cellular mutagenicity, and the present invention was thereby completed.

That is, the present invention provides a histostain composition for endoscopy containing one or more members selected from colors derived from Monascus.

The present invention also provides a diagnostic method with an endoscope, which includes administering a composition containing one or more members selected from Monascus-derived colors and observing, with an endoscope, tissue stained with the composition.

Further, the present invention provides use of one or more members selected from Monascus-derived colors in production of a staining agent for endoscopy.

According to the histostain composition of the present invention, the surface of an affected region and the interior of tissue can be simultaneously visualized by observation under a visible light or with a confocal endoscope, that is, without removing tissue. The histostain composition of the invention is excellent in operationality because it can be distributed from the digestive tract. Because a natural color is used, the composition is highly safe for the human body.

### BRIEF DESCRIPTION OF THE DRAWINGS

FIG. 1 shows absorption and fluorescence spectra of Monascus color (red).

FIG. 2 is a photograph showing the observation result of the rat large intestine stained with Monascus color (red) (63-power objective lens). In the figure, 1) is an image of a surface part, 2) is an image in a depth of 5.98 μm, 3) is an image in a depth of 11.96 μm, and 4) is an image in a depth of 17.94 μm.

FIG. 3 is a sectional image of the rat large intestine stained with Monascus color (red) (with a 10-power lens).

FIG. 4 is a visible light endoscopic image of the rat small intestine stained with Monascus color (red). The upper image is a stained image, and the lower is an un-stained image.

FIG. 5 is a photograph showing the observation result of the rat large intestine stained with Monasco Yellow (objective lens: 63-power immersion lens).

FIG. 6 is a photograph showing the observation result of the rat large intestine stained with Monasco Red (objective lens: 63-power immersion lens).

FIG. 7 is a graph showing a change in the fluorescence intensity of Monasco Yellow at different pH values.

FIG. 8 is a photograph showing the observation result of the rat large intestine stained with fluorescein.

FIG. 9 is a photograph showing a stained image of the mouse large intestine 1 minute after spreading with Monasco Yellow.

FIG. 10 is a photograph showing an image of the removed mouse large intestine stained with Monasco Yellow.

FIG. 11 is a photograph showing a stained image of the mouse large intestine 10 minutes after spreading with Monasco Yellow.

FIG. 12 is a photograph showing a stained image of the mouse large intestine 60 minutes after spreading with Monasco Yellow.

FIG. 13 is a photograph showing a stained state of the large intestine upon perfusion of Monasco Yellow through the heart in a mouse.

FIG. 14 is a photograph showing a state of the large intestine upon perfusion of Sodium fluorescein through the heart in a mouse.

FIG. 15 is a photograph showing an image (20-power lens) of the mouse large intestine stained with xanthomonasin A.

FIG. 16 is a photograph showing an image (63-power immersion lens) of the mouse large intestine stained with xanthomonasin A.

FIG. 17 is a photograph showing a normally stained image (40-power lens) of the mouse large intestine stained with hematoxylin-eosin.

FIG. 18 is a photograph showing a fluorescently stained image (63-power lens) of the mouse large intestine stained with xanthomonasin A.

FIG. 19 is a photograph showing an image (1 minute after staining) taken, under a confocal microscope, of the mouse large intestine stained with xanthomonasin A.

FIG. 20 is a photograph showing an image (10 minutes after staining) taken, under a confocal microscope, of the mouse large intestine stained with xanthomonasin A.

### DESCRIPTION OF THE PREFERRED EMBODIMENT

The endoscope of the present invention includes medical endoscopes such as a gastrointestinal endoscope, respiratory endoscope, vascular endoscope, joint endoscope, peritoneal endoscope, and the like. Among these endoscopes, the gastrointestinal endoscope is particularly preferable. In the present invention, the visible light endoscope includes every endoscope used in observation under a visible light and includes a usual endoscope, a magnifying endoscope (10- to 200-power), and a dye spraying endoscope for observing a visible light. The fluorescent endoscope includes an endoscope for measuring fluorescence generated by irradiation with exciting light, for example a magnifying fluorescent endoscope. The confocal endoscope refers to an endoscope having a confocal system. The confocal endoscope has both a usual monitoring optical system and a confocal monitoring optical system.

The histostain composition for an endoscope according to the present invention includes one or more members selected from colors derived from Monascus. Monascus is Ascomycota Monascus, and is not particularly limited insofar as it belongs to the genus Monascus, and examples thereof include Monascus pilosus, Monascus anka, Monascus perpureus and the like. The colors derived from Monascus include the ones represented by the following formula (1) to (5); ankaflavin (represented by formula (1) wherein R¹=C₇H₁₅), monascin (formula (1) wherein R¹ =C5H₁₁) monascorubrin (formula (2) wherein R²=C₇H₁₅), rubropunctatin (formula (2) wherein R²=C₅H₁₁), monascorubramine (formula (3) wherein R³=C₇H₁₅, R⁶=H), rubropunctamine (formula (3) wherein R³=C₅H₁₁), rubropunctalysine (formula (3) wherein R³=C₅H₁₁, R⁶=(CH₂)₄CH(NH)COOH), and xanthomonasin or derivatives thereof (formulae (4) or (5) wherein each of R⁴ and R⁵ is C₅H₁₁ or C₇H₁₅) and one or more kind(s) selected therefrom are preferably contained in the stain composition of the present invention. The xanthomonasin of the formula (4) or (5) is xanthomonasin A when R⁴ and R⁵ are C₅H₁₁ or xanthomonasin B when R⁴ and R⁵ are C₇H₁₅. 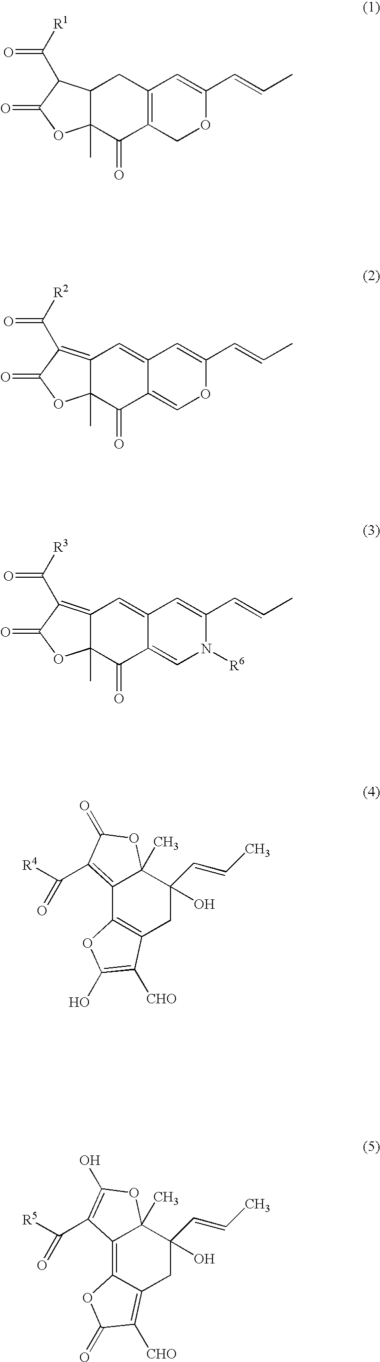
wherein R¹, R², R³, R⁴ and R⁵ each represent a C1 to C11 alkyl group, preferably C₅H₁₁ or C₇H₁₅, and R⁶ represents a hydrogen atom or —(CH₂)ₙCH(NH₂)COOH wherein n is a number of 2 to 6, preferably 4.

As the components described above, one or more members selected from ankaflavin, monascorubrin, monascorubramine, and xanthomonasin or derivatives thereof are particularly preferably contained in the stain composition of the present invention.

These colors derived from Monascus are red or yellow dyes, and they have been conventionally used in fish cakes and flavored octopus in Japan and used in fermented foods such as koshu (one kind of port wine) and beni tofu (red bean curd) since ancient times in China, thus indicating that they do not have a problem with regard to safety. The LD₅₀ of Monascus colors orally administered to mice is not less than 20 g/kg, and no-observed-adverse-effect-level in repeat-dose studies (13 weeks) is 1.25 g/kg/day.

These colors derived from Monascus can be obtained by extraction of Monascus microorganisms with, for example, water-containing ethanol, water-containing propylene glycol, or acidic ethanol with hydrochloric acid, at room temperature to at slightly increased temperature.

Commercial products of these colors derived from Monascus include, for example, Sun Red M, Sun Red MA, Sun Red MR and Sun Yellow No. 1244 manufactured by San-Ei Gen F.F.I., Inc.; Monasco A, Monasco G, Monasco Z, Monasco RX, TS Red MP, TS Yellow M and TS Yellow MP manufactured by Taisho Technos Co., Ltd.; Monasco Red AL450RA and Monasco Yellow S manufactured by KIRIYA Chemical Co., LTD.; KC Red MR and KC Red MY-2 manufactured by Kobe Chemical Co., LTD.; and Monascus Colors manufactured by Wako Pure Chemical Industries, Ltd.

The content of the Monascus-derived color in the histostain composition of the present invention is preferably 0.01 to 70 mass %, more preferably 0.01 to 50 mass %, still more preferably 0.01 to 20 mass %, from the viewpoint of staining property and the vividness of a stained image.

The histostain composition of the present invention can be used in the form of liquid, granules, tablets and the like. The histostain composition is preferably liquid for spreading in the digestive tract or for submucous administration, or is preferably liquid, granules, tablets and the like for oral administration.

The histostain composition of the present invention can be compounded with a wide variety of ingredients, depending on its form (drug form). For example, the histostain composition can be compounded with a viscous agent, a thickening agent, a surfactant, a sweetener, a preservative, a perfume, a pH adjusting agent, water and the like.

The pH adjusting agent includes those for adjusting to pH 5 to 9, for example, hydrochloric acid, phosphoric acid, citric acid, malic acid, acetic acid and salts thereof, sodium hydroxide, potassium hydroxide, sodium bicarbonate, and tetrasodium pyrophosphate.

The histostain composition can be compounded with ethanol, water and the like as the solvent. In the case of tablets, known tabletting ingredients such as a binder, a disintegrating agent and the like can be used.

The histostain composition of the present invention can stain tissue in red or similar color or in yellow or similar color, and is thus useful as an agent for staining the surface of tissue at the time of observation with a usual visible light endoscope. The endoscope used herein is a usual endoscope or a magnifying endoscope and is useful for endoscopic observation with magnification of from 10-power to 500-power under a visible light.

The Monascus-derived color, upon excitation with light in the vicinity of 487 nm, emits strong fluorescence in the vicinity of 514 nm. Accordingly, the color is used as a fluorescent dye for staining the surface of tissue for observation with a fluorescent endoscope or a confocal endoscope.

By spreading the Monascus-derived color on the digestive tract lumen, the color can penetrate easily into its tissue and is thus useful as an interstitial staining fluorescent dye with a confocal endoscope. As the confocal endoscope, there is an endoscope for example having an observation depth of 250 μm (observation field, 500 μm×500 μm; magnification, 500-power). Accordingly, this confocal endoscope can be used to obtain a fluorescent dye sectional image of internal tissue (for example, up to 250 μm in depth) after spreading or orally administering the histostain composition of the present invention.

When the endoscope utilizing a confocal optical system has both an ordinary monitoring optical system and a confocal monitoring optical system, an affected region is observed under usual light with the naked eye, and then the surface and interior of tissue in the affected region of question in the digestive tract can be diagnosed with a confocal endoscope by observing a fluorescent dye sectional image of internal tissue (for example, up to 250 μm in depth) without excising the affected tissue. That is, the shape of cell and nucleus in living tissue can be observed in a living state. As a result, the diagnosis of disorders in digestive tracts, such as precancerous state, cancer, ulcer, ulcerous colitis and the like, can be carried out safely and rapidly with less invasiveness, while accuracy can be dramatically improved.

In the endoscopic observation, the histostain composition of the present invention may be applied directly to the digestive tract lumen or may be submucously or orally administered.

### EXAMPLES

Hereinafter, the present invention is described in more detail, but the present invention is not limited to these examples.

### Example 1

A wide variety of naturally occurring dyes were measured for their absorption spectra and fluorescence excitation spectra to identify fluorescent substances. The measured dyes were purchased from Wako Pure Chemical Industries, Ltd., except Monascus color (yellow). Measurement was carried out as follows: Each dye was dissolved at a concentration of 1 to 0.1 mg/mL in water to prepare a solution. The absorption maximum wavelength of the dye was determined by measuring the absorbance continuously at wavelengths of 200 to 600 nm by a spectrophotometer (BioSpec-1600 manufactured by SHIMADZU CORPORATION) thereby determining the wavelength at which the absorption maximum appears. Each dye was irradiated with a light of absorption maximum wavelength as exciting light, and the wavelength of scattering light detected in a direction perpendicular to the axis of the exciting light was determined as fluorescence maximum absorption by a fluorescence spectrophotometer (RF-1500 manufactured by SHIMADZU CORPORATION).

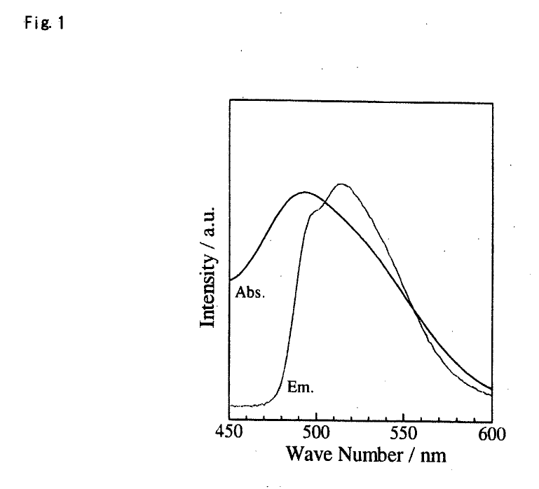

As shown in Table 1, a shift (stokes shift) toward longer wavelength rather than the excitation wavelength used for fluorescence (scattering light) wavelength was observed in Monascus color (yellow) and Monascus color (red). No stokes shift was observed in the dyes other than the Monascus colors. FIG. 1 shows absorption and fluorescence spectra of Monascus color (red). Monascus dye (red) used was Monascus color manufactured by Wako Pure Chemical Industries, Ltd. As Monascuscolor (yellow), Monasco Yellow manufactured by KIRIYA Chemical Co., LTD. was used.

TABLE 1

| | Excitation | Fluorescence (light | |
|---|---|---|---|
| Naturally occurring | maximum | scattering) maximum | Stokes |
| dyes | wavelength | wavelength | shift* |
| Cochineal dye | 497 nm | 497 nm | 0 |
| Lac die | 491 nm | 491 nm | 0 |
| Grape skin color | 536 nm | 536 nm | 0 |
| Annatto dye | 331 nm | 331 nm | 0 |
| Beat red | 534 nm | 534 nm | 0 |
| Cacao dye | 473 nm | 473 nm | 0 |
| Monascus color | 487 nm | 514 nm | 27 |
| (red) | | | |
| Monascus color | 460 nm | 503 nm | 43 |
| (yellow) | | | |

*Stokes shift: fluorescence maximum wavelength − excitation maximum wavelength

### Example 2

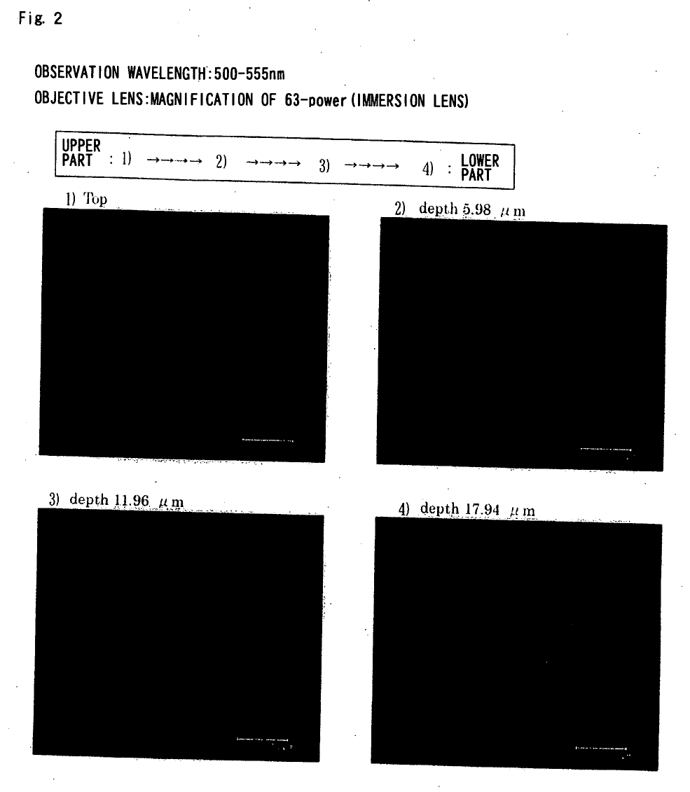

The rat large intestine fixed with a formalin solution was cut into pieces of 5 by 5 mm square and washed with phosphate buffered physiological saline (137 mmol/l NaCl, 8.1 mmol/l Na₂HPO₄, 2.7 mmol/l KCl, 1.5 mmol/l KH₂PO₄; abbreviated hereinafter as PBS(−)). The tissue was placed in an aqueous solution (10 mg/mL) of Monascus color (red) (Monascus color manufactured by Wako Pure Chemical Industries, Ltd.), then left for 1 minute, and washed with PBS (−) for 10 seconds. Thereafter, the tissue was fixed with a formalin solution and observed with a confocal microscope (TCS SP2 manufactured by Leica; hereinafter, the confocal microscope used in the Examples refers to this confocal microscope). The tissue was observed with fluorescence at wavelengths of 500 to 535 nm by excitation with a 488-nm Ar laser. FIG. 2 shows confocal microphotographs. These microphotographs are images of the same section photographed every about 6 μm toward inside (inner ward) from the surface layer. As shown in FIG. 2, large intestine is stained, and it was found that the fluorescent stained image of the tissue which is stained inside can be obtained.

### Comparative Example 1

When the naturally occurring dyes (annatto dye, grape skin color, beat red, cochineal dye) not emitting fluorescence were used and observed with a confocal microscope in the same manner as in Example 2, no fluorescent stained image could be obtained.

### Example 3

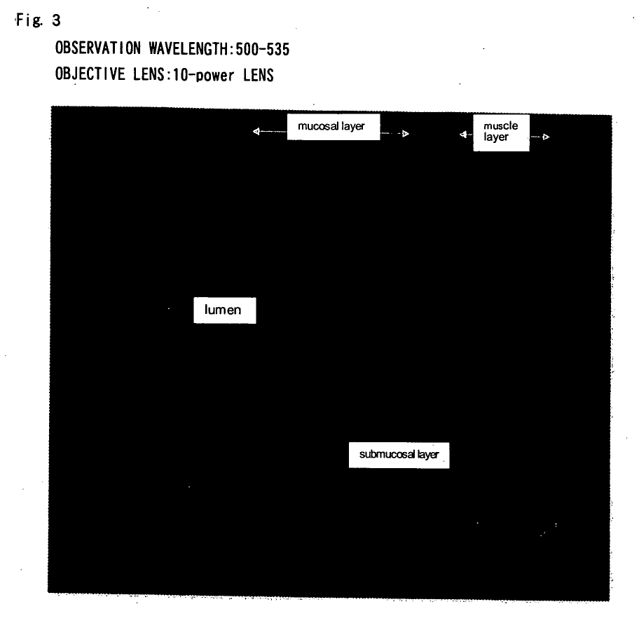

From the large intestine stained in Example 2, a thinly sliced section sample was prepared. The sample was observed with a confocal microscope for fluorescence at wavelengths of 500 to 535 nm by excitation with a 488-nm Ar laser. As a result, it was found that an image wherein the large intestine was stained uniformly from the lumen side to the fascias side (excluding the submucosal layer) can be obtained as shown in FIG. 3. From the photograph, it was found that the depth of stain with the Monascus color was 500 to 1000 μm or more.

### Example 4

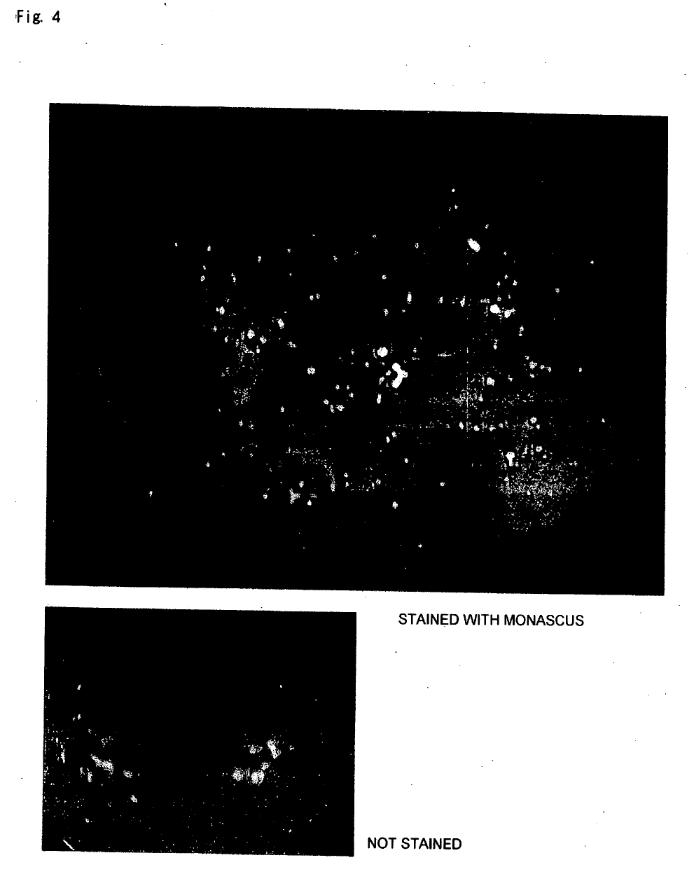

The rat small intestine fixed with a formalin solution was cut into pieces of 10 by 10 mm square, coated with an aqueous solution (10 mg/mL) of Monascus color (red) (Monascus color manufactured by Wako Pure Chemical Industries, Ltd.) and observed with a visible light endoscope. As a result, the small intestine was stained in red as shown in FIG. 4, and information regarding, for example, the shape of villi which is hardly judged in a non-stained observation image could be obtained more vividly.

### Example 5

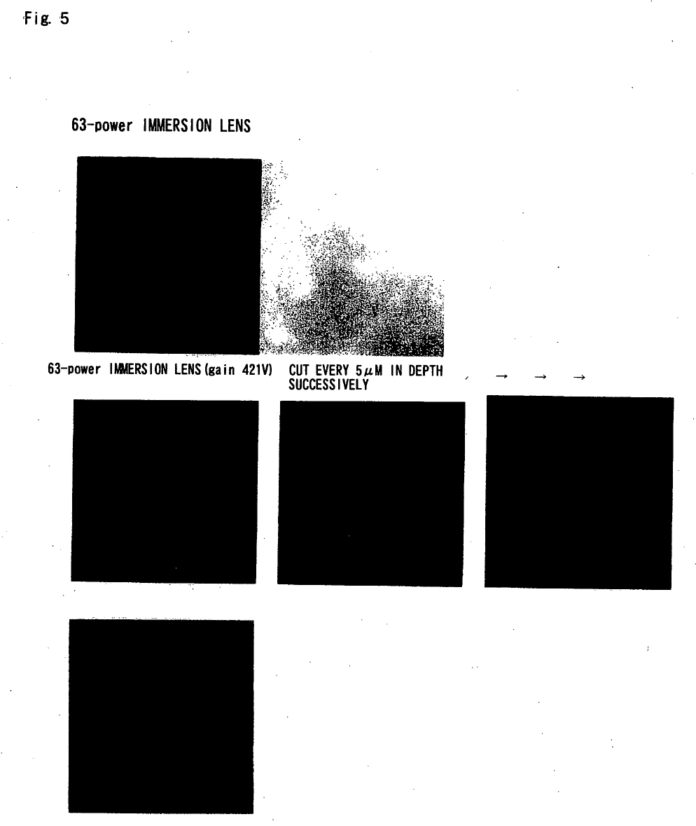

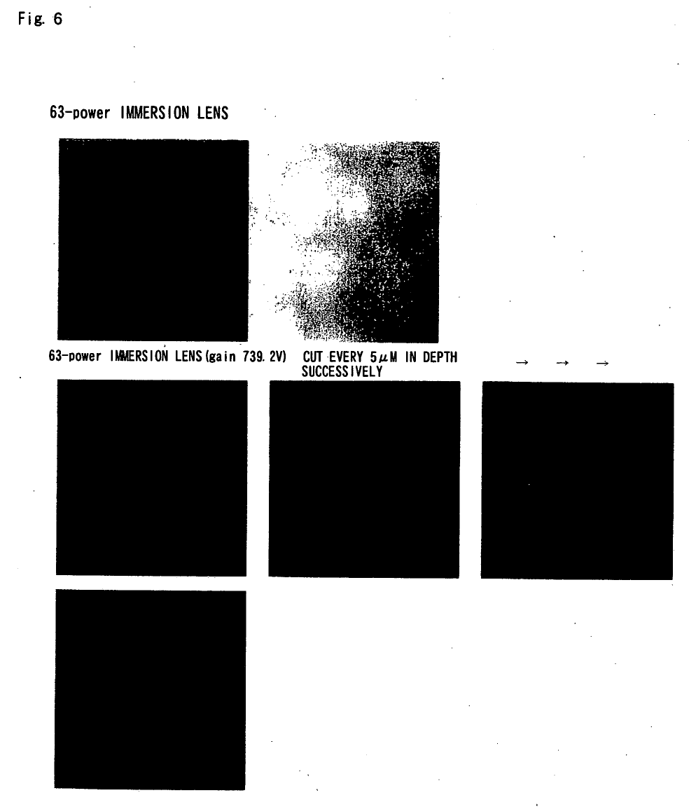

A rat large intestine staining test was carried out with an aqueous solution (6 mg/mL) of Monascus color (yellow) (Monasco Yellow S manufactured by KIRIYA Chemical Co., LTD.) and an aqueous solution (10 mg/mL) of Monascus color (red) (Monasco Red 9000P manufactured by KIRIYA Chemical Co., LTD.). As the sample, the formalin-fixed rat large intestine was dipped in each color solution for 1 minute and observed with a confocal microscope. The results indicated that as shown in FIGS. 5 and 6, the staining solutions of both Monasco Red and Monasco Yellow penetrated into the interior of the tissue, and strong fluorescence was exhibited. As a result of observation, it was found that only the cytoplasm was vividly stained, while the cell nuclei were not stained.

### Example 6

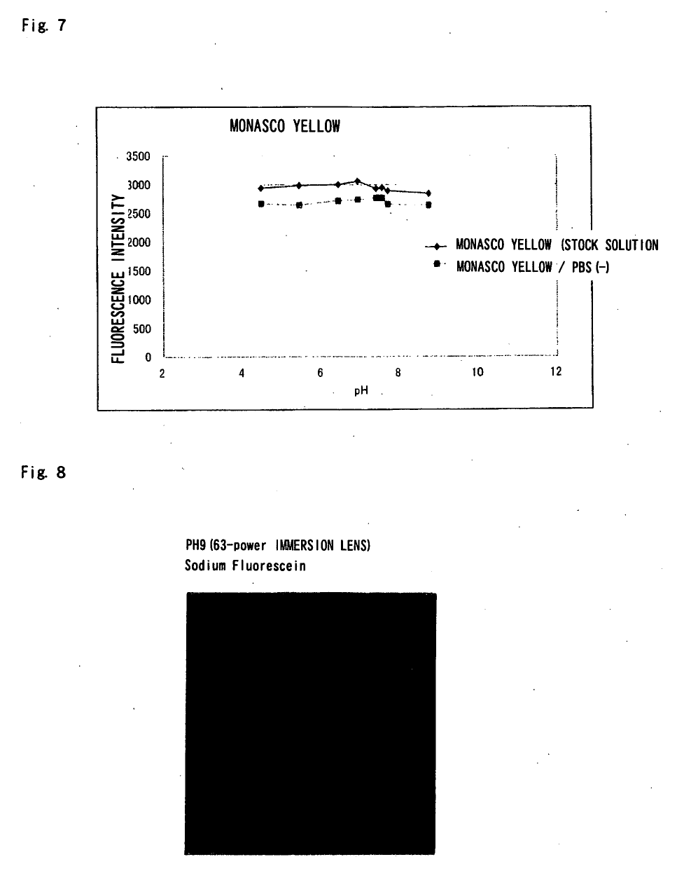

Change in staining property at different pH values upon in vivo staining is a very important factor. Accordingly, the influence of pH on the fluorescence property of Monasco Yellow S (manufactured by KIRIYA Chemical Co., LTD.) was examined. The results are shown in FIG. 7. The fluorescence intensity was not significantly changed in measurement in buffer solutions at; pH 4.65, 5.00, 6.00, 6.80, 7.00, 7.40, 8.00, and 9.30. It was thus found that the fluorescence property of the Monascus yellow color is not significantly influenced by pH.

### Comparative Example 2

The rat large intestine was observed under a confocal microscope in the same manner as in Examples 2 and 5 except that fluorescein which is adjusted to pH 9 was used in place of the Monascus color. The result indicated that as shown in FIG. 8, the tissue was stained but the fluorescence intensity of fluorescein caused a higher background, which made the observation difficult.

### Example 7

The staining effect of spreading Monascus color (yellow) on the digestive tract lumen in the living body was verified by the following method.

Monasco Yellow (manufactured by KIRIYA Chemical Co., LTD.) (0.1 mg/mL, 500 μl) was injected through the anus into a mouse (8-week-old, male), and 1 minute later, the large intestine was removed and observed for its staining under a confocal microscope (manufactured by Leica).

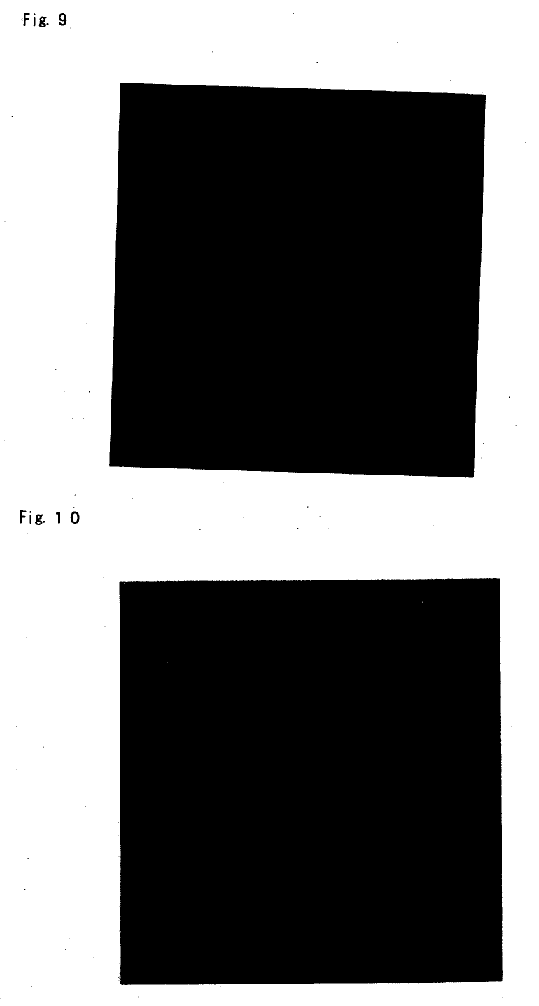

The mucosal tissue on the surface of the mouse large intestine from the living body was stained excellently by spreading the color (FIG. 9). The cells present in the mucosal tissue on the large intestine include fibroblasts and white blood cells in the lamina propria mucosa, in addition to columnar epithelial cells and goblet cells. Cytoplasmic components in these cells were stained with Monasco Yellow, but mucosal components in the goblet cells, and nuclei of all the cells, were poor in stainability.

These results were in accordance with those in a microscopic image (FIG. 10) obtained by in vitro staining of the tissue of the removed large intestine.

### Example 8

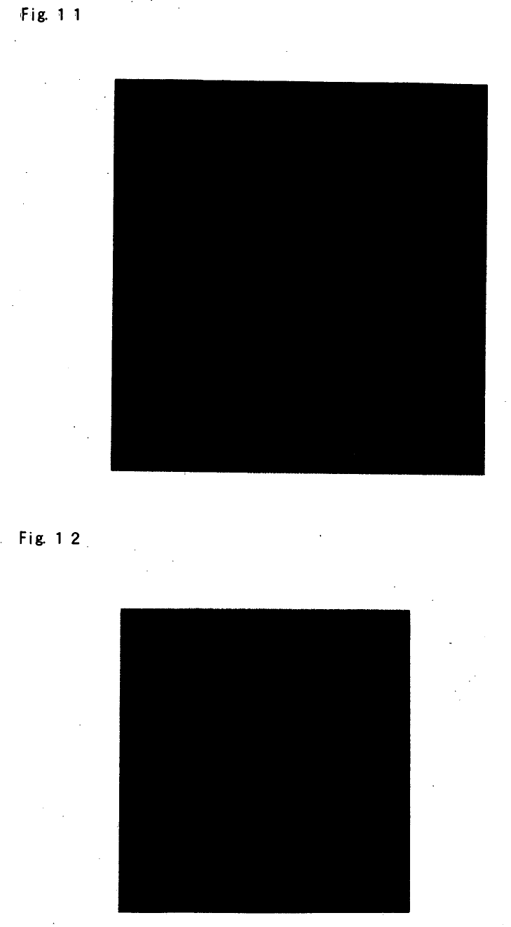

The large intestines in living mice were stained with Monasco Yellow (manufactured by KIRIYA Chemical Co., LTD.) in the same manner as in Example 7 and the mice were maintained 10 and 60 minutes after staining and then observed for change in staining property with time. The observation was carried out in the same manner as in Example 7. The sites stained well with Monasco Yellow did not change regardless of the time in which the mice had been maintained after administration, but the brightness of fluorescence of the tissue had been lowered at the time when the observation was made after 60 minutes of rearing (FIGS. 11 and 12).

By judging the results comprehensively, the well-stained sites of the large intestine mucosa are summarized in Table 2.

TABLE 2

| | Cytoplasm | Nucleus | Other |
|---|---|---|---|
| Mucus | N/A | N/A | − |
| Columnar epithelium | + | − | N/A |
| Goblet cell | + | − | − (Mucus) |
| Lamina propria mucosa cell | + | − | N/A |

−: not stained,
N/A: Not applicable,
+: stained

### Comparative Example 3

Difference in Fluorescence Level

In a confocal microscope (manufactured by Leica), spectral sensitivity can be regulated so as to set the brightness of displayed fluorescence at the same level. That is, this function can be used for relative estimation of the fluorescence intensity of each of the samples having different brightness levels.

The fluorescence brightness of a sample with Sodium fluorescein relative to Monascus color (yellow), as calculated on the basis of such spectral sensitivity as to give the same degree of fluorescence brightness, was 0.74 times after 10 minutes or 0.84 times after 60 minutes.

Accordingly, when the sample was stained with a solution of the fluorescent dye at the same concentration, it can be said that the fluorescence brightness of the large intestine tissue is higher when stained with the Monascus color (yellow) than with Sodium fluorescein.

### Example 9

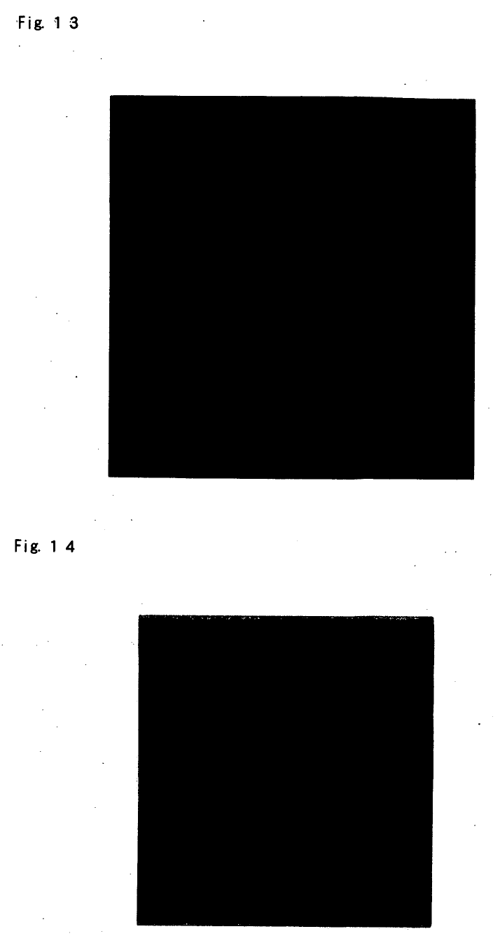

A staining test was carried out with Monascus color (yellow) (1 mg/mL, 2 mL) by perfusion thereof through the heart of a mouse. As a result of staining, the large intestine tissue was stained excellently. The permeability of stain by this perfusion staining method was higher than the method in which the excised tissue is stained (Example 2) or the method in which colors are injected through the anus (Example 7). In observation of the lumen with a confocal microscope, the cytoplasm of almost all cells constituting the mucosa were well stained, while the mucus components of goblet cells, and cell nuclei, were poor in stainability (FIG. 13). That is, the well-stained sites were the same as in Examples 7 and 8.

### Comparative Example 4

Sodium fluorescein (1 mg/mL) was also examined in the same manner as in Example 9.

Sodium fluorescein gave the same stained image as in Example 9, namely, many of the cells constituting the mucosal tissue were stained well, a mucus portion of goblet cells was not stained. The cell nuclei could not be judged with respect to the stained state (FIG. 14).

These results were in accordance with those in the image obtained as a result of staining by the spreading method, and better results in respect of staining range, density etc. could be obtained by the perfusion staining method.

For the purpose of observing the state of cells and the shape of nuclei, observation with Monascus color (yellow) can be said to give more useful data than by Sodium fluorescein.

### Example 10

Monascus yellow was subjected to high speed liquid chromatography (SCL10A manufactured by SHIMADZU CORPORATION), and its major components, i.e., xanthomonasin A and xanthomonasin B, were extracted and purified.

Monascus yellow was injected into an ODS column (Wakosil 25C18) and then separated with a mobile phase of 20% acetonitrile/water. On the basis of the resulting chromatogram, components in each peak were recovered, concentrated with an evaporator and subjected again to chromatography under the same conditions as above. Each fraction was analyzed for purity and mass by LC-MS (Acquity UPLC-ZQ manufactured by Waters), and those fractions showing a single peak on the chromatogram and having the same mass as that of xanthomonasin A (or xanthomonasin B) were concentrated and designated as purified xanthomonasin A (or xanthomonasin B).

### Example 11

Using purified xanthomonasin A in Example 10, a mouse large intestine staining test was carried out.

A mouse (ddY, 9-week-old, male) was anesthetized, and 100 μL xanthomonasin A (centrifuged and dried sample, 10 mg/mL saline) was injected via an injection needle into the large intestine lumen for staining.

After 5 minutes, the mouse large intestine was excised and its confocal image was taken and observed under a confocal microscope (TCS SP2).

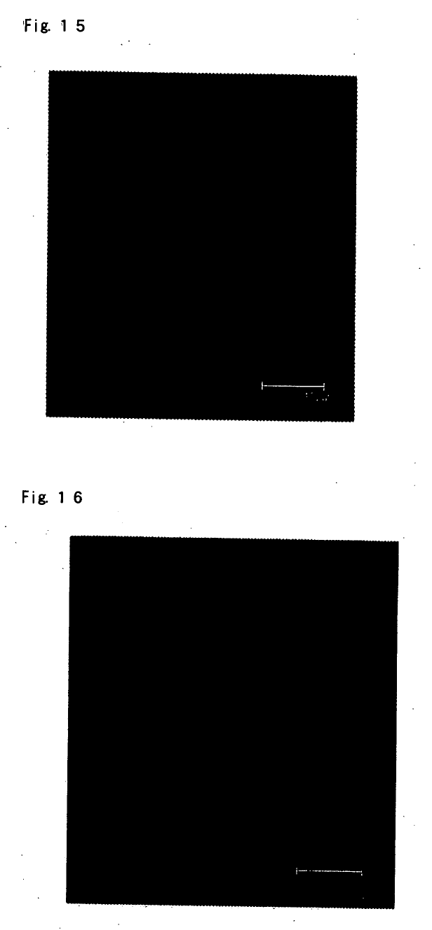

FIG. 15 shows the image taken through a 20-power lens. FIG. 16 shows the image taken through a 63-power immersion lens. The gain values indicating the gain were 348.2 V and 339.7 V, and very vivid sectional images were obtained with xanthomonasin A.

### Example 12

Purified xanthomonasin A in Example 10 was used in a mouse large intestine staining test and for observing a frozen section.

A mouse (ddY, 11-week-old, male) was anesthetized, and 100 μL xanthomonasin A (centrifuged and dried sample, 10 mg/mL saline) was injected via an injection needle into the large intestine lumen for staining.

After 5 minutes, the mouse large intestine was excised, frozen and embedded in OCT compound, and the frozen section thus obtained was cut into thin slices each having a thickness of 6 μm. The thin slices were observed by hematoxylin-eosin staining and also observed for fluorescence by staining with xanthomonasin A.

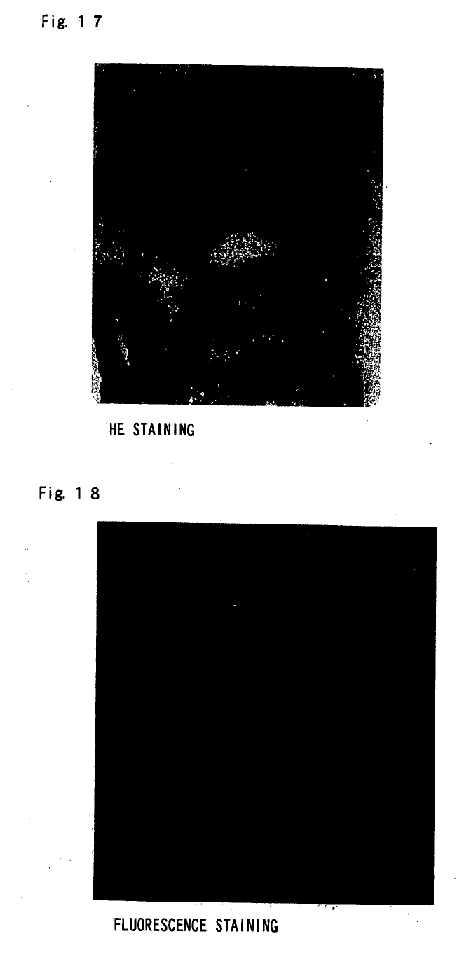

FIG. 17 shows the hematoxylin-eosin-stained image taken through a 40-power lens, and FIG. 18 shows the xanthomonasin A-stained fluorescent image taken through a 63-power lens.

The two stained images indicated that epithelial cells were stained relatively excellently by staining with xanthomonasin A, and a muscular plate was also excellently stained. The gain value, which indicates a gain, was in the range of 300 to 500 V, indicating excellent staining property.

### Example 13

Mice (ddY, 9-week-old, male) were anesthetized, and 100 μL xanthomonasin A (centrifuged and dried sample, 1 mg/mL saline) was injected via an injection needle into the large intestine lumen for staining.

The mouse large intestines were excised after 1 minute and 10 minutes respectively, and the samples were observed under a confocal microscope (TCS SP2).

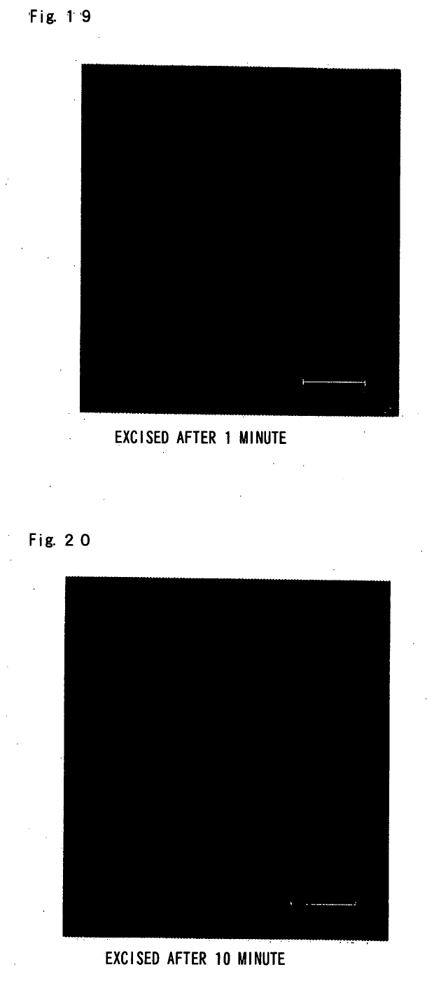

FIG. 19 shows the confocal image of the large intestine excised 1 minute after staining, and FIG. 20 shows the confocal image of the large intestine excised 10 minutes after staining. The two images exhibit differences with time in staining property and permeation, but were identical in respect of stained sites. It can be said that when the large intestine was stained for 10 minutes, the visibility could be further improved.

What is claimed is:

1. A diagnostic method with an endoscope, which comprises administering a composition containing one or more members selected from colors derived from Monascus and observing, with the endoscope, tissue stained with the composition.

2. The diagnostic method according to claim 1, wherein the colors derived from Monascus comprise one or more members selected from compounds represented by the following formulae (1) to (5): 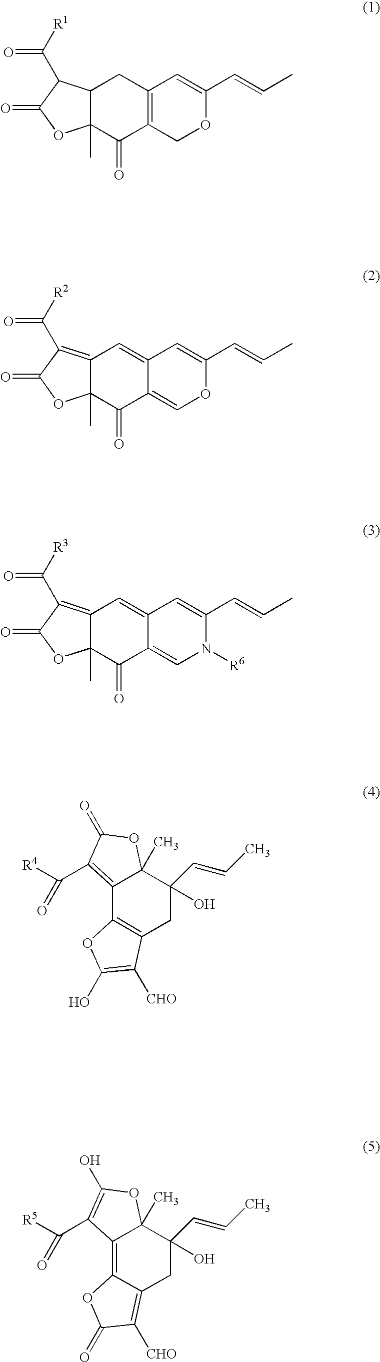
wherein R¹, R², R³, R⁴ and R⁵ each represent a C1 to C11 alkyl group, and R⁶ represents a hydrogen atom or —(CH₂)ₙCH(NH₂)COOH wherein n is a number of 2 to 6.

3. The diagnostic method according to claim 1, wherein the colors derived from Monascus are one or more members selected from the group consisting of ankaflavin, monascin, monascorubrin, rubropunctatin, monascorubramine, rubropunctatin, rubropunctalysine and xanthomonasin.

4. The diagnostic method according to claim 1, wherein the endoscope is an endoscope for medical use.

5. The diagnostic method according to claim 1, wherein the endoscope is a visible light endoscope, a fluorescent endoscope or a confocal endoscope.

6. The diagnostic method according to claim 1, wherein administration of the composition is carried out by oral administration, direct administration to the digestive tract or submucous administration.

7. The diagnostic method according to claim 1, which is for staining the surface of a digestive tract lumen and/or the interior of cells in a digestive tract lumen.

8. A histostain composition for an endoscope comprising one or more members selected from colors derived from Monascus.

9. The stain composition according to claim 8, wherein the colors derived from Monascus comprise one or more members selected from compounds represented by the following formulae (1) to (5): 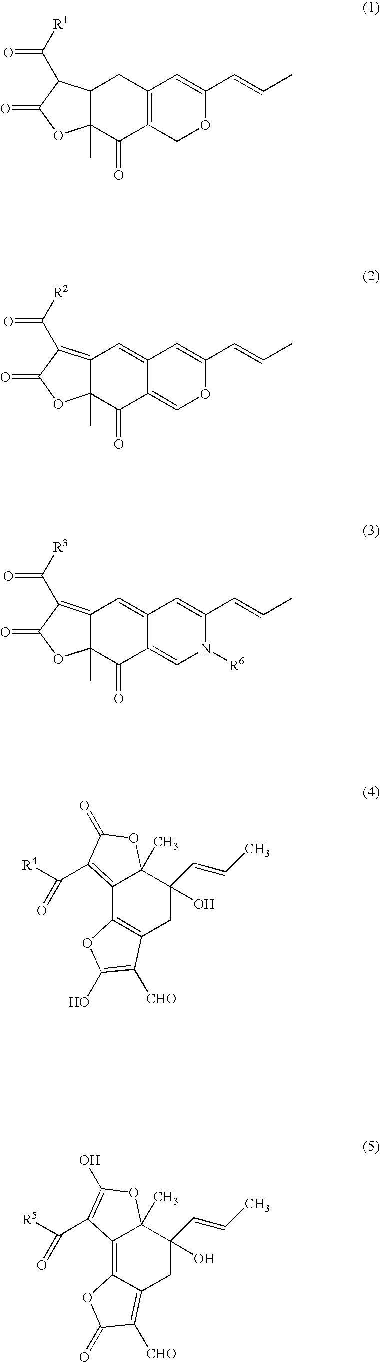
wherein R¹, R², R³, R⁴ and R⁵ each represent a C1 to C11 alkyl group, and R⁶ represents a hydrogen atom or —(CH₂)ₙCH(NH₂)COOH wherein n is a number of 2 to 6.

10. The stain composition according to claim 8, wherein the colors derived from Monascus are one or more members selected from the group consisting of ankaflavin, monascin, monascorubrin, rubropunctatin, monascorubramine, rubropunctatin, rubropunctalysine and xanthomonasin.

11. The stain composition according to claim 8, wherein the endoscope is an endoscope for medical use.

12. The stain composition according to claim 8, wherein the endoscope is a visible light endoscope, a fluorescent endoscope or a confocal endoscope.

13. The stain composition according to claim 8, which is orally administered, directly administered to the digestive tract, or submucously administered.

14. The stain composition according to claim 8, which is for staining the surface of a digestive tract lumen and/or the interior of cells in a digestive tract lumen.
